(19) United States
(12) Patent Application Publication
(10) Pub. No.: US 2021/0000370 A1
(43) Pub. Date: Jan. 7, 2021

(54) In-Ear EEG Sensor Using Malleable Form Fitting Foam

(71) Applicant: X Development LLC, Mountain View, CA (US)

(72) Inventors: Russell Mirov, Los Altos, CA (US); Phillip Yee, San Francisco, CA (US); Jonathan Berent, Mountain View, CA (US)

(21) Appl. No.: 16/855,133

(22) Filed: Apr. 22, 2020

Related U.S. Application Data
(60) Provisional application No. 62/869,637, filed on Jul. 2, 2019.

(51) Int. Cl. A61B 5/0478; A61B 5/00; A61B 5/04; A61B 5/01; A61B 5/024; A61B 5/053; A61B 5/145

(52) U.S. Cl. CPC A61B 5/0478; A61B 5/0006; A61B 5/04008; A61B 5/01; A61B 2562/0219; A61B 5/0531; A61B 5/14532; A61B 2562/06; A61B 5/024

(57) ABSTRACT

The technology relates to fabricating and employing a sensor unit with a malleable housing to obtain various bio-signals detected while the sensor unit is disposed in the wearer's ear canal. A set of sensor contacts is arranged along an exterior ear-contacting portion of the housing. The malleable material can be compressed for insertion into the ear canal, then automatically expanding to contact the ear canal at multiple points. The sensor contacts are distributed along the exterior of the housing to provide an orientation agnostic configuration. The sensor unit is able to be worn for hours, a day or longer. During wear, the contacts can detect EEG and/or MEG-related signal, such as Alpha waves. Other sensors may be included with the sensor unit to supplement the detection of bio-signals. The obtained signals may be processed on-board or transmitted to a remote device for off-board processing.

### CROSS-REFERENCES TO RELATED APPLICATIONS

The present application claims the benefit of the filing date of U.S. Provisional Application No. 62/869,637, filed Jul. 2, 2019, the entire disclosure of which is incorporated by reference herein.

### BACKGROUND

Wearable sensors have been used to detect electroencephalogram (EEG) and other bio signals from the wearer's body. These signals can be used for medical or non-medical (e.g., brain control interface) purposes. In the past, caps have been worn on the head to capture EEG signals. These caps can capture input via multiple data channels. However, wearing a cap for an extended period of time can be cumbersome and uncomfortable. It can also be difficult to get high quality signals, especially if the wearer has thick hair. In-ear sensors, for instance using custom-molded earpieces, have also been considered. These devices may have a rigid hearing aid type arrangement fabricated using a cast shape or a digital scan of the wearer's ear canal, which can be costly and time consuming. More recently, malleable sensors with one or more electrical contacts have been proposed, which attempt to locate electrodes at particular points in the ear and are tied to specific medical applications. Such approaches suffer from various drawbacks, including difficulty providing multiple high quality in-ear contacts, partially or completely blocking ambient sounds, and costly and labor-intensive manufacturing techniques.

### BRIEF SUMMARY

The technology relates to a universal in-ear sensor unit with multiple electrical contacts, which can be used to obtain EEG and other signals.

According to one aspect, a sensor assembly configured for partial or complete insertion in an ear of a wearer is provided. The sensor assembly comprises a malleable housing, a plurality of electrically conductive contacts, and a flexible printed circuit. The malleable housing has a first end, a second end remote from the first end, an exterior surface and an interior cavity extending along a longitudinal axis between the first end and the second end. The plurality of electrically conductive contacts is arranged along the exterior surface of the housing. The plurality of contacts are spaced apart along the longitudinal axis or radially along the exterior surface. The flexible printed circuit is at least partly received within the housing. The flexible printed circuit includes a plurality of leads configured to receive bio signals from corresponding ones of the plurality of electrically conductive contacts.

In one example, the malleable housing is configured to compress during insertion into an ear canal of the wearer, and to at least partly expand after the insertion to make contact along multiple points within the ear canal along the exterior surface of the housing. In another example, at least some of the plurality of contacts circumscribe the exterior surface of the housing.

In one configuration the plurality of contacts includes a set of ring-shaped contacts. Here, each ring-shaped contact may have a width of between 1-5 mm. Additionally or alternatively, the set of ring-shaped contacts may be spaced apart from any adjacent neighbors by at least 1 mm. The plurality of contacts may include an end contact along the first end of the housing. And in one arrangement, each of the ring-shaped contacts has a same width.

According to a further example, a first set of the plurality of contacts circumscribe the exterior surface of the housing along a first section thereof and a second set of the plurality of contacts are distributed along a second section of the exterior surface of the housing.

In yet another example, the interior cavity extends entirely from the first end to the second end and is configured to remain open upon insertion into an ear canal of the wearer and to pass ambient sounds from an external environment through the sensor assembly. The interior cavity may be formed as one or more holes in the housing. The interior cavity may comprise one or more tubes received within the housing. In this case, the one or more tubes may form a rigid or semi-rigid structure to prevent a collapse of the interior cavity.

The plurality of contacts may each comprise AgCl.

The sensor assembly may further comprise a plurality of traces disposed within the housing. In this case, each of the plurality of traces has a first end coupling to one of the plurality of contacts and a second end coupling to the flexible printed circuit.

The sensor assembly may also comprise an on-board processing system including one or more processors configured to process received bio signals.

The sensor assembly may also include one or more sensors selected from the group consisting of a temperature sensor, a heart rate sensor, an electrodermal activity sensor, a pulse oximeter sensor, a glucometer, an accelerometer, an orientation sensor and a location sensor.

A sensor system configured to detect and process bio signals of a wearer may include any configuration of the sensor system described herein, and also include a remote processing system having a transceiver configured for communication with a transceiver of the sensor assembly. Here, the sensor system also includes one or more processors configured to process the bio signals received from the sensor assembly.

According to another aspect of the technology, a method of fabricating a malleable sensor assembly for in-ear use is provided. The method comprises attaching a flexible printed circuit to a malleable housing, the flexible printed circuit including wiring having a plurality of electrodes extending therefrom; arranging the electrodes to contact selected points on an exterior surface of the housing; applying a mask over the exterior surface of the housing to cover some regions and expose others; performing a coating process to apply a conductive material to any exposed regions; and removing the mask.

The method may further include defining an interior cavity extending along a longitudinal axis between a first end of the housing and a second end of the housing. Here, at least a portion of the flexible printed circuit is disposed within the interior cavity.

### BRIEF DESCRIPTION OF THE DRAWINGS

FIGS. 1-2 illustrate features of a sensor unit configured for use with aspects of the technology.

FIG. 3 illustrates an example sensor unit in accordance with aspects of the technology.

FIGS. 4A-B illustrate additional example sensor units in accordance with aspects of the technology.

FIG. 5 illustrates a cutaway view of a sensor unit in accordance with aspects of the technology.

FIG. 6A illustrates an in-ear sensor assembly in accordance with aspects of the technology.

FIG. 6B illustrates an external processing system in accordance with aspects of the technology.

FIG. 7 illustrates an exemplary flexible printed circuit for use with an in-ear sensor system.

FIGS. 8A-B illustrate a step of assembling the exemplary flexible printed circuit of FIG. 7 with an in-ear sensor system according to aspects of the technology.

FIGS. 9A-B illustrate another step of assembling the exemplary flexible printed circuit with an in-ear sensor system according to aspects of the technology.

FIGS. 10A-B illustrate a further step of assembling the exemplary flexible printed circuit with an in-ear sensor system according to aspects of the technology.

FIG. 11 illustrates an example method in accordance with aspects of the technology.

### DETAILED DESCRIPTION

Features of the technology include a sensor unit with a malleable housing configured to obtain EEG and/or other bio-signals detected while the unit is disposed in the wearer's ear canal. The sensor unit has a set of sensor contacts arranged along the exterior ear-contacting portion of the housing. For instance, the housing may comprise a foam or otherwise malleable material that can be compressed for insertion into the ear canal, automatically expanding to contact the ear canal at multiple points where the contacts are arranged. The contacts may be configured so that the sensor unit is orientation agnostic, which enables the wearer to insert the unit into his or her ear without worrying about its exact positioning. The unit may be worn for hours, a day or longer.

In addition or alternative to use as an EEG sensor unit, the in-ear sensor assembly may be used to detect magnetoencephalograph (MEG) signals. For instance, Alpha waves on the order of 8-12 Hz can be detected either by either EEG or MEG. In addition, lower frequency signals (e.g., Delta waves between 0.5-3 Hz or Theta waves between 3-8 Hz) and/or higher frequency signals (e.g., Beta waves between 12-38 Hz or Gamma waves between 38-42 Hz) may also be detected. One or more of these types of signals can be evaluated and analyzed either alone or in conjunction with other data to provide information (e.g., biomarkers) about the wearer. The other data may be obtained by additional in-ear sensors (e.g., in the same assembly or in a sensor assembly worn in the other ear) or sensors located elsewhere on or near the wearer. These may include heart rate and temperature sensors. Electrodermal activity (EDA) sensors that detects skin potential, resistance, conductance, admittance, or impedance, such as galvanic skin response sensors, may also be employed. Furthermore a pulse oximeter sensor, a glucometer, orientation sensors, location sensors and/or accelerometers can also be used. These sensors can be used in any combination. The biomarkers or other information can be evaluated to help classify mental or emotional states, as well as activities of daily living.

### Example Implementations

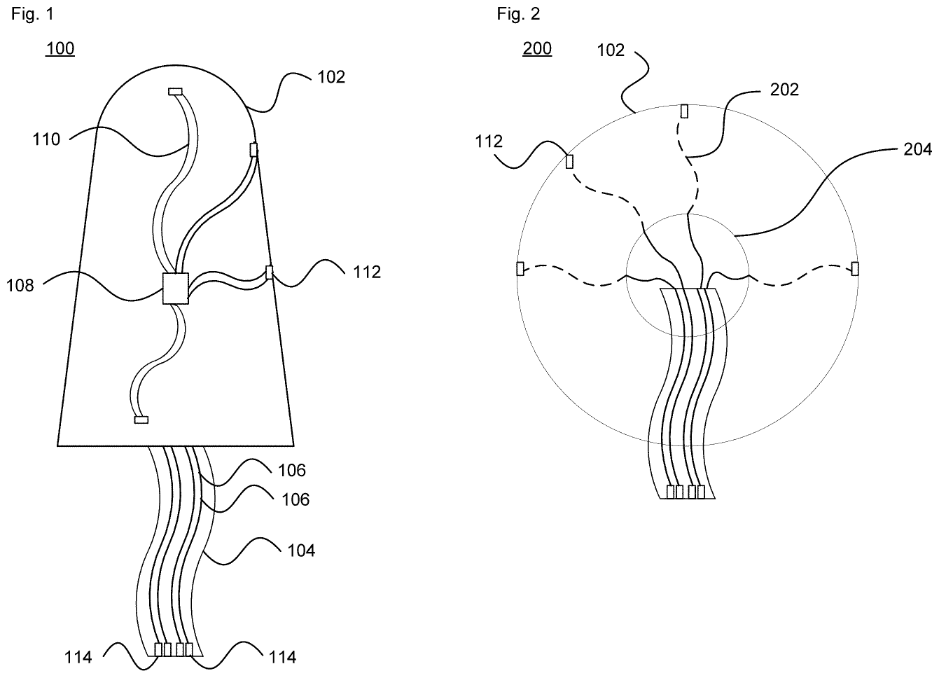

Various configurations of an in-ear sensor assembly will now be described in accordance with aspects of the technology. FIG. 1 illustrates an exemplary in-ear housing 102 having a flexible printed circuit (FPC) 104 mounted therein. The housing 102 comprises a foam or otherwise malleable housing that can be compressed for insertion into the ear canal, automatically expanding to contact the ear canal at multiple points. FPC 104 includes multiple conductive wires 106. In one scenario, the conductive wires 106 extend within the housing 102 and branch off from a common section 108. Branches 110 each terminate at a respective electrode 112. Signals detected by the electrodes 112 are passed to contacts 114 for off-board (remote) processing. As discussed further below, on-board signal processing and data collection via additional sensors may also be performed by the in-ear sensor assembly.

The conductive wires should be arranged to minimize interference (e.g., cross-coupling) with neighboring wires. The frequencies of interest are very low (e.g., below 50 Hz), which minimizes crosstalk. Nonetheless, a ground plane may be incorporated in the FPC so that the conductive wires act as are transmission lines instead of unshielded wires.

FIG. 2 illustrates a partial cutaway end view 200 of the housing 102 and FPC 104. This view 200 is from the end which will abut a portion of the external auditory canal during use. As shown by dashed lines 202, the wires of branches 110 extend at least partly through the housing 102, terminating at the electrodes 112. Also shown in this view is opening 204, which extends longitudinally either substantially or completely through the housing 102. The opening 204 is configured to pass sound from the external environment through the sensor assembly with little or no attenuation or distortion.

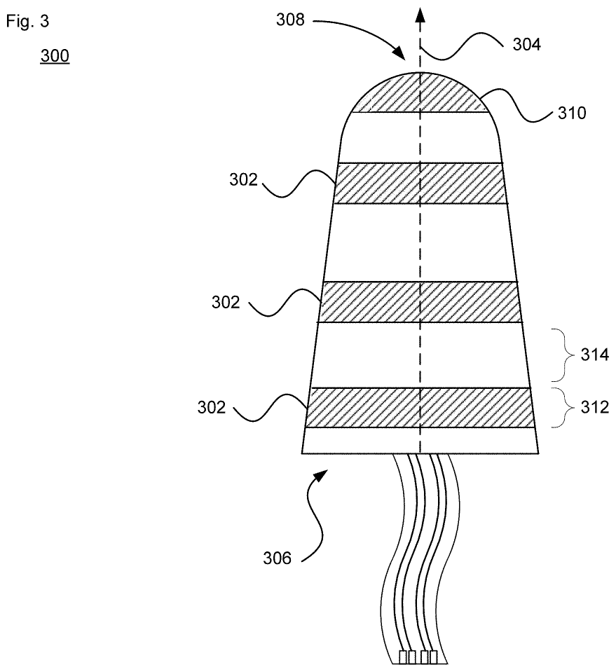

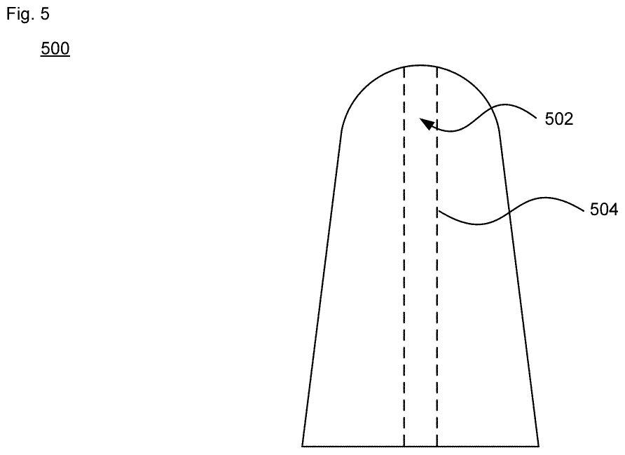

The approach in this case provides an orientation-agnostic in-ear sensor assembly with multiple electrical contacts in, for instance, a ring-type or other distributed arrangement as seen in FIGS. 3 and 5A-B, which are discussed below. As noted above, the housing 102 may be comprised of foam, although other types of materials may be employed. In the example of FIGS. 1-2, the foam is non-conductive. Alternatively, another malleable material that is configured to have conductive regions can be used. Such conductive regions may be arranged so that the electrodes can pass signals through the regions to the FPC. By way of example, carbon particles or some other conductive material (e.g., silver particles) may be added to portions of the malleable material to form the conductive regions. For a foam-type arrangement, a conductive coating is applied to selected portions of the housing's ear-contacting outer surface. The coating may be silver chloride (AgCl) or another conductive material suitable for use with skin-contacting electrodes arranged on a malleable housing.

FIG. 3 illustrates an example electrode arrangement 300 employing a series of conductive rings 302. As shown, the conductive rings 302 are positioned on the exterior surface of the housing. The rings are spaced apart along the longitudinal axis 304 of the sensor assembly between a first end 306 and a second end 308. Here, the first end 306 corresponds to the end that will abut a portion of the external auditory canal during wear, and the second end 308 is arranged closest to the tympanic membrane (ear drum) upon insertion. Each ring 302 is associated with a corresponding electrode connector (not shown) to provide separate sensed signals to the on-board and/or off-board processing system. Also shown in FIG. 3 is another conductive element 310 disposed along the second end 308, which is also associated with its own electrode connector (not shown). The conductive element 310 may have a different shape from the rings 302, such as arcuate, hemispherical, semicircular, circular or some other geometric shape.

The number and spacing of the rings may vary. In one scenario, as many rings as possible are provided so long as the rings do not short one another or generate interfering signals, and are able to obtain reliable high quality signals that are not duplicative of signals from neighboring rings. By way of example only, each ring may have a width 312 of between 2-5 mm, or more or less, and the rings may have a spacing 314 of at least 2-5 mm apart. In another scenario, the rings and/or other conductive element(s) have thinner widths (e.g., no more than 1-2 mm) and spacings (e.g., no more than 1-2 mm apart) to ensure that a sufficient number of rings make contact with different parts of the ear canal and/or provide a minimum signal to noise ratio (e.g., 10 dB, 20 dB or more or less). Here, if certain elements do not provide signals of selected quality, the data received from those elements may be discarded by the on-board or remote processing system.

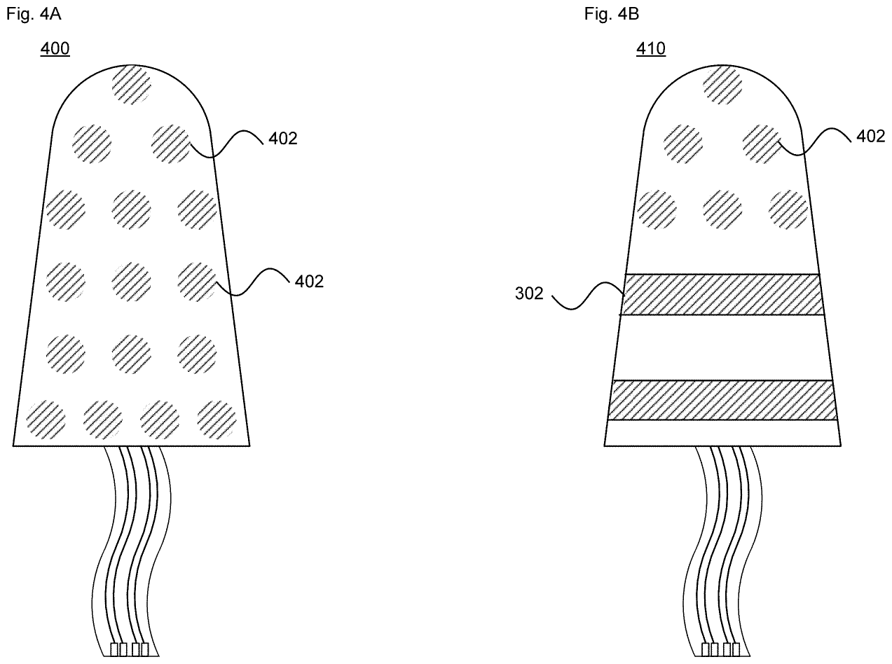

In other examples, any or all of the contacts may have non-ring shapes, so long as the contacts circumscribe the outer surface of the housing or otherwise provide sufficient signal coverage. FIG. 4A illustrates one example 400 where a series of dots or other shapes 402 are distributed along the exterior surface of the housing. And FIG. 4B illustrates another example 410 with a combination of rings 302 and dots 402. These and other electrode shapes may be distributed longitudinally and/or radially along the exterior of the housing.

The result for any of the above configurations is an orientation agnostic in-ear sensor assembly that does not require the wearer to insert the device in any particular orientation in the ear canal. Nonetheless, the device may include one or more physical reference features so that the wearer may more easily place it at the same clocking orientation each time it is worn, which can aid repeatability for sensing signals.

It may be desirable to allow the wearer to hear ambient sounds while the sensor unit is worn. This will avoid the sensation of the device being an ear plug, and will be more conducive to wearing for an extended period of time (e.g., hours or days). To achieve this, the sensor unit includes one or more holes or tubes extending generally along the longitudinal axis for sound to pass through. FIG. 5 illustrates a cutaway view 500 of one example. Here, a generally cylindrical hole 502 defined by sidewall(s) 504 extends substantially or completely through the housing.

In one example, the hole(s) is formed as part of the malleable housing, and remains open after insertion into the ear canal. In another example, one or more tubes of a non-collapsible (rigid or semi-rigid) material are inserted into or fabricated as part of the housing. The tubes prevent pinching or crimping of the foam or other malleable housing material, allowing the wearer to hear ambient sounds without appreciable distortion (e.g., without cutting off or attenuating higher frequencies beyond 10-15 kHz) or reduction in volume. In a further example, in place of or in addition to the hole(s), a small speaker may be incorporated into the malleable housing. In this scenario, the speaker would provide sound to the inner portions of the ear canal. The speaker can emit sounds in place of or to augment sounds passed through the hole(s).

### Example Operation

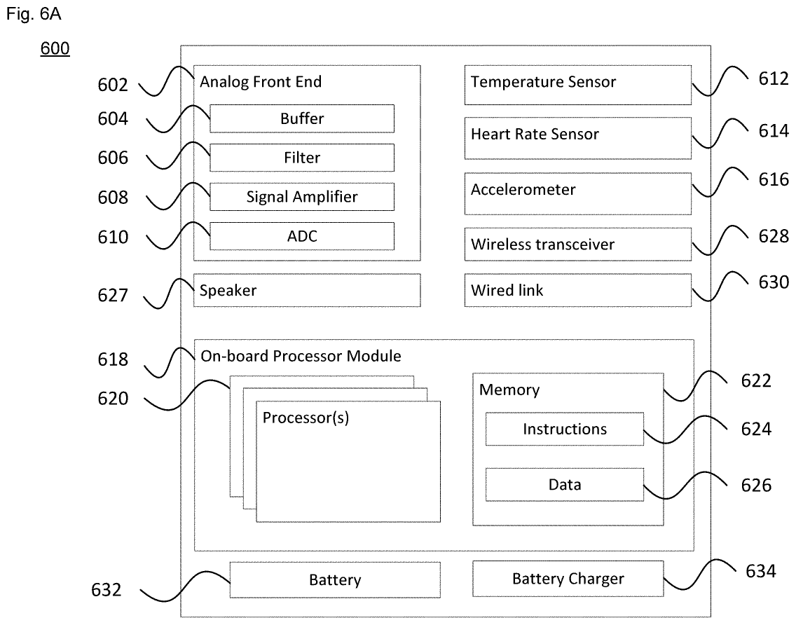

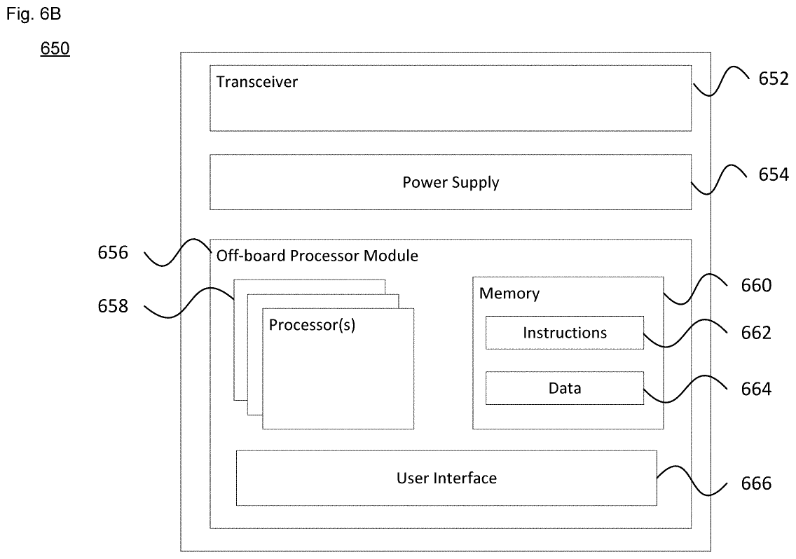

Upon insertion into the ear canal, the sensor assembly is configured to detect Alpha waves or other waves. Processing of such signals may be performed at the sensor assembly, by a remote processing system, or both. FIG. 6A illustrates one example of an on-board processing system 600, and FIG. 6B illustrates one example of a remote processing system 650. With regard to FIG. 6A, the signals from the electrodes (e.g., rings 302 and/or dots 402) may first be received by an analog front end (AFE) 602. The AFE 602 may provide one or more of signal buffering via buffer 604, filtering via filter(s) 606, signal amplification by amplifier 608, and/or analog to digital conversion by analog to digital converter (ADC) 610.

The processing system 600 may also receive biometric and other information from additional sensors, such as a temperature sensor 612, a heart rate sensor 614 and an accelerometer 616. While not illustrated, as noted above other sensors may include EDA sensors such as galvanic skin response sensors, a pulse oximeter sensor, a glucometer, as well as orientation sensors and/or location sensors. Some or all of this information may also be processed by AFE 602.

At this point, the processing system 600 may analyze the obtained data with an on-board processor module 618, which includes one or more processors 620 as well as memory 622 that stores instructions 624 and data 626 that may be executed or otherwise used by the processor(s) 620. The one or more processors 620 may be, e.g., a controller or CPU. Alternatively, the one or more processors 620 may be a dedicated device such as an ASIC, FPGA or other hardware-based device. The memory 622 may be of any type capable of storing information accessible by the processor(s) in a non-transitory manner, such as solid state flash memory or the like.

The instructions 624 may be any set of instructions to be executed directly (such as machine code) or indirectly (such as scripts) by the processor(s). For example, the instructions may be stored as computing device code in the non-transitory memory. In that regard, the terms “instructions” and “programs” may be used interchangeably herein. The instructions may be stored in object code format for direct processing by the processor(s), or in any other computing device language including scripts or collections of independent source code modules that are interpreted on demand or compiled in advance. The data 626 may be retrieved, stored or modified by one or more processors in accordance with the instructions 624. As an example, data 626 may include heuristics to be used when calibrating or evaluating electrode viability, for instance to rank electrode suitability based on signal-to-noise ratio or other metrics.

As noted above, in one example a speaker 627 may be incorporated into the malleable housing. The speaker 627 is operatively coupled to the on-board processor module 618 to provide sound to the inner portions of the canal. The module 618 may actuate the speaker 627 to supplement (augment) sounds passed through the hole(s) extending through the malleable housing, or to generate different sounds such as audible cues (e.g., tones) to provide information or give aural feedback to the wearer.

Alternatively or in addition to on-board signal analysis, the processing system may transmit the obtained data to remote processing system 650. This may be done, for instance, via a wireless transceiver 628 or a wired link 630, such as I2C, SPI, Universal Asynchronous Receiver/Transmitter (UART), I2S, or some other low-signal count communications path. In the latter case, the FPC may extend out the end of the sensor assembly and be physically coupled to remote processing system 650 that can receive and/or process the obtained bio signals. Alternatively, in the former case the wireless transceiver the FPC may communicate with the remote processing system 650 via Bluetooth™, Bluetooth™ LE, near field communication (NFC) or some other wireless communication method.

System 600 also includes a battery 632 to power the components of the processing system. It may also include a battery charger 634. The battery charger may be contactless, or may be plugged into an external power source to charge the battery. The system 600 may be incorporated into or mounted on the FPC. Alternatively, some or all of the system 600 may be received within the housing and operatively coupled to the FPC as needed for receiving sensor data and/or transmitting information to the remote processing system 650.

Turning to FIG. 6B, as shown remote processing system 650 includes a transceiver 652. The transceiver 652 is configured to communicate with one or both of wireless transceiver 628 and wired link 630. The system 650 also includes a power supply 654, which may include batteries and/or a connection for an outlet or the like. The information received from the on-board processing system 600, whether raw or unprocessed, is passed from the transceiver 652 to the off-board processor module 656.

The off-board processor module 656 is configured to analyze the obtained data with one or more processors 658 as well as memory 660 that stores instructions 662 and data 664 that may be executed or otherwise used by the processor(s) 658, in a manner similar to described above. The one or more processors 620 may be, e.g., a controller or CPU. Alternatively, the one or more processors 620 may be a dedicated device such as a DSP, an ASIC, FPGA or other hardware-based device. The memory 622 may be of any type capable of storing information accessible by the processor(s) in a non-transitory manner, such as solid state flash memory, hard disc, optical medium or the like.

The off-board processor module 656 also includes a user interface subsystem 666, which may be used to present information regarding the processed data to the earpiece wearer, a technician, doctor or other authorized user.

### Example Fabrication Process

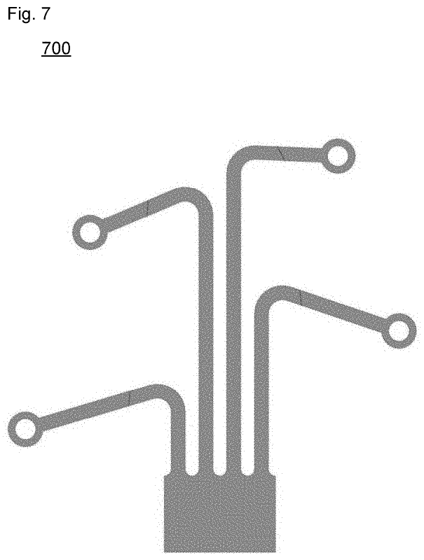

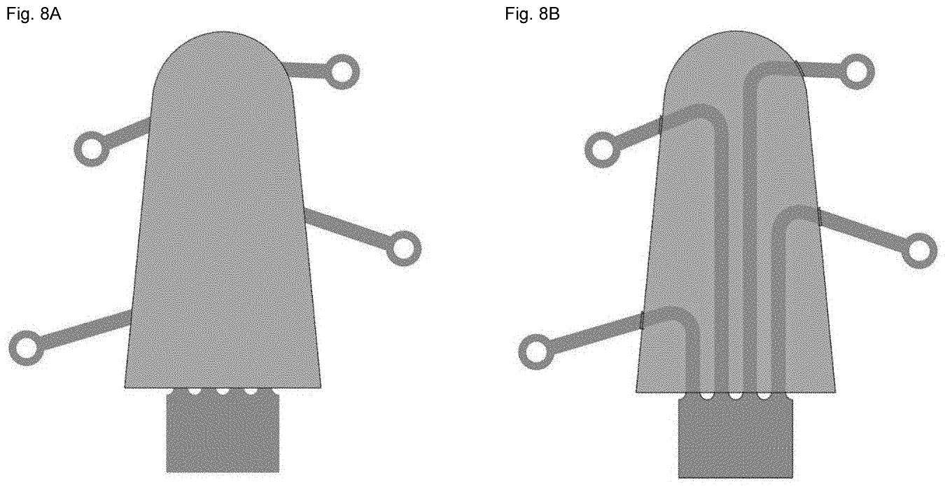

FIGS. 7-10B illustrate one example for fabricating a malleable in-ear sensor assembly. FIG. 7 illustrates an FPC pattern 700. FIG. 8A illustrates the FPC pattern 700 incorporated within the housing, and FIG. 8B illustrates a cutaway view showing the branches extending out of the housing. In one scenario, a portion of the FPC pattern with the branches may be inserted into a central hole of the housing, and the branches may be pushed through the housing to exit at different locations. Alternatively, the malleable material may be fabricated around the FPC pattern.

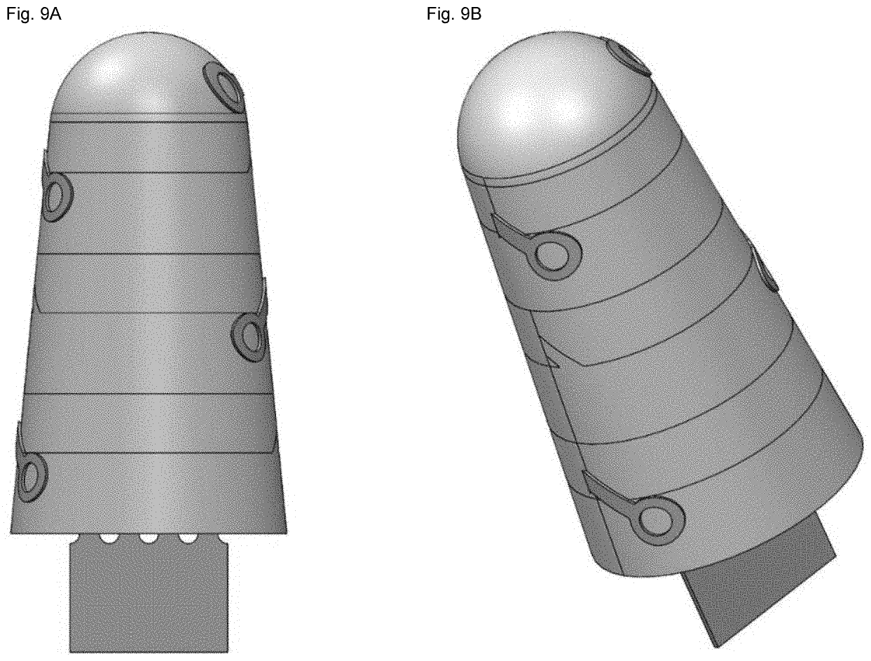

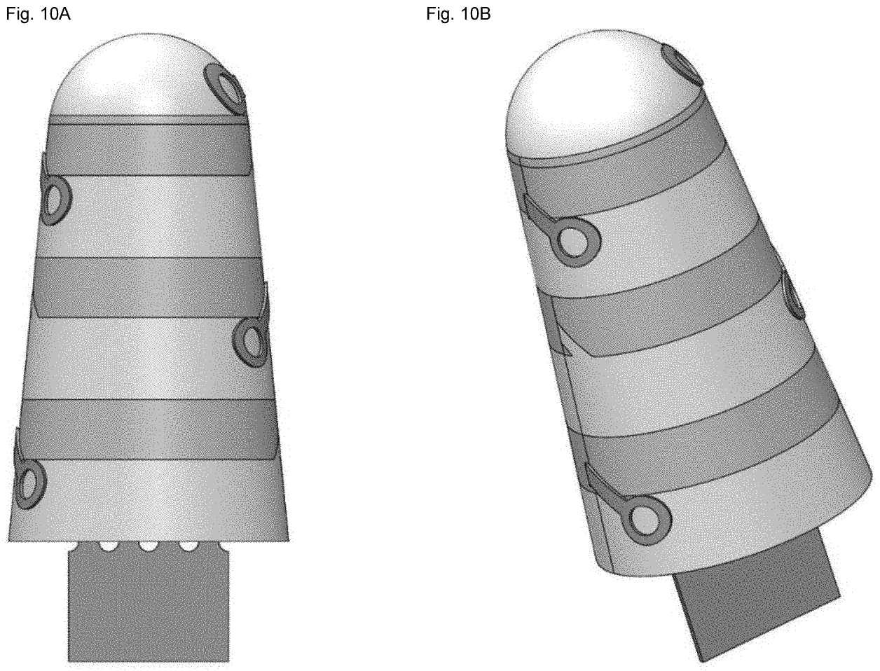

As shown in the side view of FIG. 9A and the perspective view of FIG. 9B, the electrodes at the ends of each branch are arranged to contact selected parts of the exterior surface of the malleable housing. The conductive elements (e.g., rings, dots, or other geometric shapes) may be formed on the housing surface as follows. A mask is applied to the outer surface of the housing, in which specific regions for the rings and/or other conductive elements are left exposed. A coating process is performed, in which a conductive material is applied over the housing (e.g., dipping, spraying or printing). The conductive material may be AgCl or another material chosen for biocompatibility, low half-cell potential, malleability and compatibility with the application process. As shown in the side and perspective views of FIGS. 10A and 10B, the mask is then removed, leaving the conductive material in the rings or other patterns, which are connected to corresponding traces.

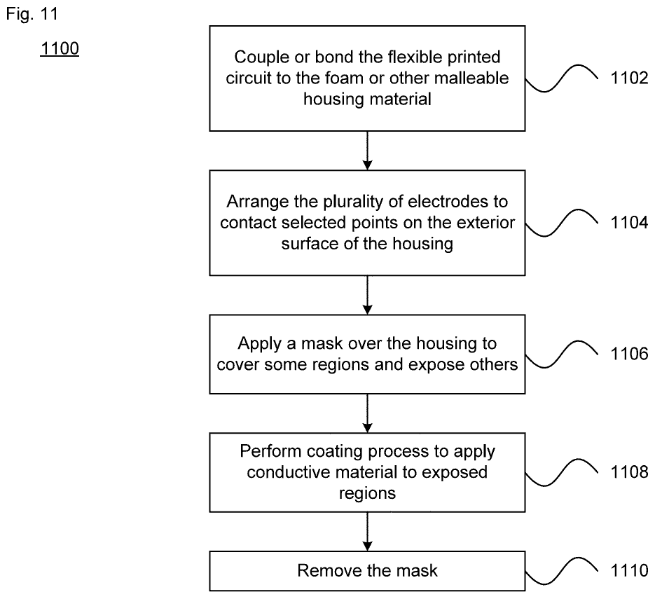

This process is also illustrated in flow diagram 1100 of FIG. 11. In particular, at block 1102 the FPC is coupled or bonded to the malleable housing material. At block 1104, the electrodes are arranged to contact selected points on the exterior surface of the housing. At block 1106, the mask is applied over the housing surface to cover some regions and expose others, for instance with ring patterns, dot patterns and/or other geometric shapes. Then at block 1108 the coating process applies the conductive material to the exposed regions. And at block 1110 the mask is removed, resulting in an orientation-agnostic in-ear sensor assembly.

Unless otherwise stated, the foregoing alternative examples are not mutually exclusive, but may be implemented in various combinations to achieve unique advantages. As these and other variations and combinations of the features discussed above can be utilized without departing from the subject matter defined by the claims, the foregoing description of the embodiments should be taken by way of illustration rather than by way of limitation of the subject matter defined by the claims. In addition, the provision of the examples described herein, as well as clauses phrased as “such as,” “including” and the like, should not be interpreted as limiting the subject matter of the claims to the specific examples; rather, the examples are intended to illustrate only one of many possible embodiments. Further, the same reference numbers in different drawings can identify the same or similar elements. The processes or other operations may be performed in a different order or simultaneously, unless expressly indicated otherwise herein.

1. A sensor assembly configured for partial or complete insertion in an ear of a wearer, the sensor assembly comprising:
a malleable housing having a first end, a second end remote from the first end, an exterior surface and an interior cavity extending along a longitudinal axis between the first end and the second end;
a plurality of electrically conductive contacts arranged along the exterior surface of the housing, the plurality of contacts being spaced apart along the longitudinal axis or radially along the exterior surface; and
a flexible printed circuit at least partly received within the housing, the flexible printed circuit including a plurality of leads configured to receive bio signals from corresponding ones of the plurality of electrically conductive contacts.

2. The sensor assembly of claim 1, wherein the malleable housing is configured to compress during insertion into an ear canal of the wearer, and to at least partly expand after the insertion to make contact along multiple points within the ear canal along the exterior surface of the housing.

3. The sensor assembly of claim 1, wherein at least some of the plurality of contacts circumscribe the exterior surface of the housing.

4. The sensor assembly of claim 1, wherein the plurality of contacts includes a set of ring-shaped contacts.

5. The sensor assembly of claim 4, wherein each ring-shaped contact has a width of between 1-5 mm.

6. The sensor assembly of claim 4, wherein the set of ring-shaped contacts are spaced apart from any adjacent neighbors by at least 1 mm.

7. The sensor assembly of claim 4, wherein the plurality of contacts further includes an end contact along the first end of the housing.

8. The sensor assembly of claim 4, wherein each of the ring-shaped contacts has a same width.

9. The sensor assembly of claim 1, wherein a first set of the plurality of contacts circumscribe the exterior surface of the housing along a first section thereof and a second set of the plurality of contacts are distributed along a second section of the exterior surface of the housing.

10. The sensor assembly of claim 1, wherein the interior cavity extends entirely from the first end to the second end and is configured to remain open upon insertion into an ear canal of the wearer and to pass ambient sounds from an external environment through the sensor assembly.

11. The sensor assembly of claim 10, wherein the interior cavity is formed as one or more holes in the housing.

12. The sensor assembly of claim 10, wherein the interior cavity comprises one or more tubes received within the housing.

13. The sensor assembly of claim 12, wherein the one or more tubes form a rigid or semi-rigid structure to prevent a collapse of the interior cavity.

14. The sensor assembly of claim 1, wherein the plurality of contacts each comprise AgCl.

15. The sensor assembly of claim 1, further comprising a plurality of traces disposed within the housing, each of the plurality of traces having a first end coupling to one of the plurality of contacts and a second end coupling to the flexible printed circuit.

16. The sensor assembly of claim 1, further comprising an on-board processing system including one or more processors configured to process received bio signals.

17. The sensor assembly of claim 1, further comprising one or more sensors selected from the group consisting of a temperature sensor, a heart rate sensor, an electrodermal activity sensor, a pulse oximeter sensor, a glucometer, an accelerometer, an orientation sensor and a location sensor.

18. A sensor system configured to detect and process bio signals of a wearer, the sensor system comprising:
the sensor assembly of claim 1; and
a remote processing system including a transceiver configured for communication with a transceiver of the sensor assembly, and one or more processors configured to process the bio signals received from the sensor assembly.

19. A method of fabricating a malleable sensor assembly for in-ear use, the method comprising:
attaching a flexible printed circuit to a malleable housing, the flexible printed circuit including wiring having a plurality of electrodes extending therefrom;
arranging the electrodes to contact selected points on an exterior surface of the housing;
applying a mask over the exterior surface of the housing to cover some regions and expose others;
performing a coating process to apply a conductive material to any exposed regions; and
removing the mask.

20. The method of claim 19, further comprising defining an interior cavity extending along a longitudinal axis between a first end of the housing and a second end of the housing, at least a portion of the flexible printed circuit being disposed within the interior cavity.
